(19) United States
(12) Patent Application Publication
(10) Pub. No.: US 2017/0087361 A1
(43) Pub. Date: Mar. 30, 2017

(54) ELECTRICAL STIMULATION APPARATUS, ELECTRICAL STIMULATION METHOD, AND RECORDING MEDIUM

(71) Applicant: Panasonic Intellectual Property Management Co., Ltd., Osaka (JP)

(72) Inventors: SHINOBU ADACHI, Nara (JP); SOUKSAKHONE BOUNYONG, Nara (JP); JUN OZAWA, Nara (JP)

(21) Appl. No.: 15/261,728

(22) Filed: Sep. 9, 2016

(30) Foreign Application Priority Data
Sep. 28, 2015 (JP) 2015-189635

(51) Int. Cl. A61N 1/36; A61N 1/04; A61B 5/00

(52) U.S. Cl. CPC A61N 1/36003; A61B 5/4812; A61B 5/4836; A61N 1/0452; A61N 1/36014

(57) ABSTRACT

An electrical stimulation apparatus includes a sleep depth determiner that determines a sleep depth by using biological information of a user who is sleeping; an electrical muscular stimulation intensity determiner that determines an electrical muscular stimulation intensity on basis of the sleep depth; and an output that output electrical muscular stimulation by using electrodes arranged on a skin of the user at the electrical muscular stimulation intensity determined by the electrical muscular stimulation intensity determiner.

### BACKGROUND

1. Technical Field

The present disclosure relates to an electrical stimulation apparatus, an electrical stimulation method, and a recording medium.

2. Description of the Related Art

Muscles contract upon application of electrical stimulation to a living body by an external device. Such electrical stimulation that induces muscle contraction is called electrical muscular stimulation (EMS). There are cases (e.g., cardiac pacemaker) where stimulation is applied from electrodes inserted into a living body. However, it is assumed herein that electrical muscular stimulation is applied only from electrodes attached on a skin surface of a living body. When electrical stimulation is applied from electrodes attached on a skin surface of a living body, a motor nerve, which has a low excitation threshold, is stimulated, and the excitation is transmitted to muscles. As a result, the muscles contract. As the intensity of electrical stimulation increases, the intensity of muscle contraction also increases up to a certain level. However, a sensory nerve close to the motor nerve is also stimulated by the electrical muscular stimulation, and therefore a user feels pain in a case where the intensity of stimulation is high. In view of this, in many cases, a maximum stimulation intensity that a user can tolerate is found, and electrical muscular stimulation having an intensity that is approximately 80% or lower of the maximum stimulation intensity is used.

One of purposes of electrical muscular stimulation is to allow a user to do exercises under a little burden. WO 2009/072437 discloses a technique for inducing muscle contraction and thereby enhancing muscles by applying electrical stimulation in the form of a pulse of 4 Hz to 20 Hz to the thigh. This allows a user to do exercises without causing a burden on the heart, lungs, and joints. It is therefore considered that the technique disclosed in WO 2009/072437 is effective especially for a user having an organ disorder such as an orthopedic disease, a diabetic complication, or a cardiovascular complication.

However, muscle contraction induced by electrical muscular stimulation is transmitted as sensory information to the brain. This causes, for example, a problem that when electrical muscular stimulation is applied during sleep, sensory information inhibits user's sleep.

### SUMMARY

One non-limiting and exemplary embodiment provides an electrical stimulation apparatus that produces a training effect by using electrical muscular stimulation without hindering user's sleep.

In one general aspect, the techniques disclosed here feature an electrical stimulation apparatus including: a sleep depth determiner that determines a first sleep depth by using first biological information of a user; an electrical muscular stimulation intensity determiner that determines a first electrical muscular stimulation intensity on basis of the first sleep depth; and an output that outputs first electrical muscular stimulation by using electrodes arranged on a skin of the user at the first electrical muscular stimulation intensity.

According to the present disclosure, a first electrical muscular stimulation intensity is changed in accordance with an estimated first sleep depth of a user. It is therefore possible to realize muscle training during sleep by using electrical muscular stimulation without hindering user's sleep.

It should be noted that general or specific embodiments may be implemented as a system, a method, an integrated circuit, a computer program, a computer-readable recording medium, or any selective combination thereof. The computer-readable recording medium encompasses a non-volatile recording medium such as a CD-ROM (Compact Disc-Read Only Memory).

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a diagram schematically illustrating a relationship between an electrical muscular stimulation intensity and a sensory information intensity;

FIG. 2 is a diagram schematically illustrating a relationship between a sleep depth and a sensitivity threshold;

FIG. 3 is a diagram virtually illustrating a relationship between an electrical muscular stimulation intensity and a sensitivity threshold at each sleep depth;

FIG. 4 is a diagram illustrating a configuration of functional blocks of an electrical stimulation apparatus according to Embodiment 1;

FIG. 5 is a diagram illustrating an example of a configuration and a use environment of the electrical stimulation apparatus according to Embodiment 1;

FIG. 6 is a simplified diagram illustrating an apparatus suitably used for leg training;

FIG. 7 is a diagram illustrating an example of a first standard used in a case where biological information is a brain wave;

FIG. 8 is a diagram illustrating a relationship between a sleep depth and a characteristic of body movement;

FIG. 9 is a diagram illustrating an example of a second standard;

FIG. 10 is a flow chart illustrating a procedure of basic processes of the electrical stimulation apparatus;

FIG. 11 is a flow chart illustrating a procedure of an example of application of the processes of an electrical muscular stimulation device according to Embodiment 1;

FIG. 12 is a diagram illustrating an electrical stimulation apparatus including a biological information measuring unit that is capable of measuring both a brain wave and body movement;

FIG. 13 is a flow chart illustrating a procedure of processes performed by the electrical muscular stimulation device by using two kinds of biological information;

FIG. 14 is a diagram illustrating a configuration of functional blocks of an electrical stimulation apparatus according to Embodiment 2;

FIG. 15 is a diagram schematically illustrating a discriminating method;

FIG. 16 is a flow chart illustrating a procedure of processes performed by an electrical muscular stimulation device in consideration of a stimulation application period;

FIG. 17 is a diagram illustrating a configuration of functional blocks of an electrical stimulation apparatus according to a modification of Embodiment 2;

FIG. 18 is a flow chart illustrating a procedure of processes performed by an electrical muscular stimulation device in consideration of a stimulation application period; and

FIG. 19 is a diagram illustrating a hardware configuration common to the aforementioned electrical stimulation apparatuses.

### DETAILED DESCRIPTION

First, terms used herein are defined as follows.

The term “sleep depth” refers to a depth of user's sleep and is also called a sleep stage. It is said that there are four stages of sleep depths. As the sleep depth increases, sleep becomes deeper. A sleep depth 3 and a sleep depth 4 are states of unconsciousness.

When electrical muscular stimulation is applied by using electrodes attached on a skin surface of a user, a motor nerve is excited, and thereby muscle contraction is induced irrespective of a user's intention. This indicates that there is a possibility that electrical muscular stimulation allows a user to train his or her muscles without being aware of the stimulation (unconsciously). However, to obtain a muscle training effect, muscle contraction of a predetermined intensity or higher is needed. For example, it is said that a muscular strength can be improved by applying a load of approximately 40% of a maximum muscular strength.

However, in a waking state, a user is more sensitive to stimulation than during sleep, and it is therefore difficult to realize unconscious training in a waking state. Sensitivity to stimulation can be quantified in accordance with a standard called “sensitivity threshold”. The sensitivity threshold is a level at which the brain perceives transmission of sensory information. In a waking state, the sensitivity threshold is relatively low, and therefore a user is more sensitive to electrical muscular stimulation. It is considered that many users in a waking state perceive electrical muscular stimulation especially in a case where the electrical muscular stimulation is strong enough to produce a training effect.

The inventors of the present invention focused on a characteristic that the sensitivity threshold is higher during sleep than during a waking state and the sensitivity threshold becomes higher as sleep becomes deeper, and thought that unconscious training using electrical muscular stimulation can be realized during sleep.

In a case where electrical muscular stimulation is applied during sleep, it is essential that the electrical muscular stimulation does not inhibit user's sleep. Meanwhile, in order to increase a training effect, it is necessary to apply stronger electrical muscular stimulation. In view of this, the inventors of the present invention arrived at an electrical muscular stimulation application method in which the depth of sleep of a user is estimated and an electrical muscular stimulation intensity is adjusted in accordance with the depth of sleep.

As the electrical muscular stimulation intensity is increased, the intensity of muscle contraction also increases up to a certain level. However, in accordance with the increase in electrical muscular stimulation intensity, the intensity of sensory information fed back to the brain also increases and reaches a level at which a user feels pain and finally reaches a level at which the user cannot tolerate pain.

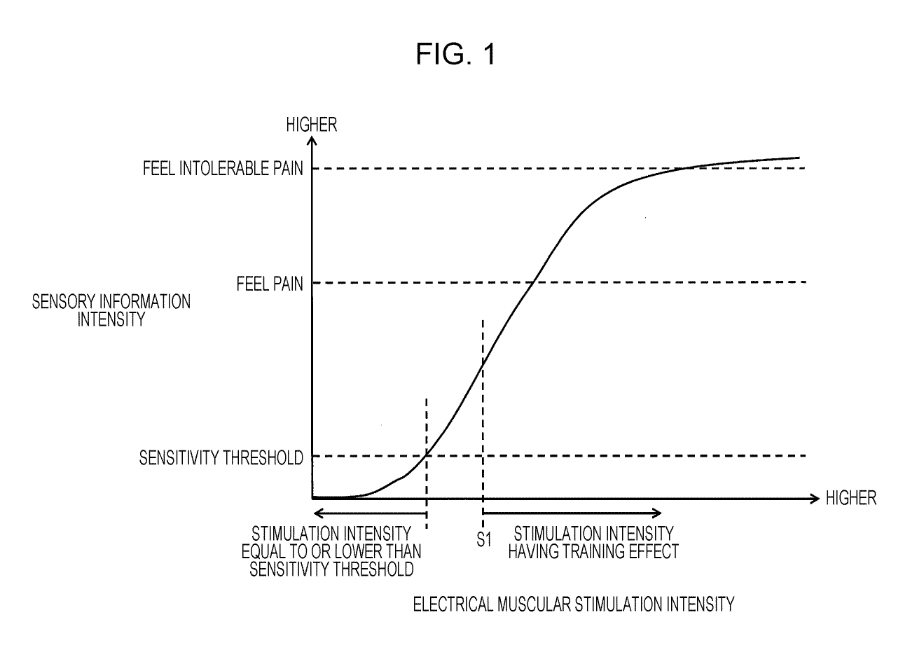

FIG. 1 schematically illustrates a relationship between an electrical muscular stimulation intensity and a sensory information intensity. The horizontal axis represents the electrical muscular stimulation intensity, and the vertical axis represents the sensory information intensity. The horizontal axis can be expressed by an electric current value in a case where an electric current source is used and can be expressed by a voltage value in a case where a voltage source is used. The vertical axis schematically illustrates a sensitivity threshold, a level at which a user feels pain, and a level at which the user cannot tolerate pain. The curve in FIG. 1 varies depending on individual or the waveform or kind of electrical muscular stimulation.

In order to produce a training effect such as muscle enlargement, muscle contraction of a predetermined intensity or higher is needed. This makes it necessary to apply electrical muscular stimulation of a predetermined intensity or higher. A stimulation intensity S1 illustrated in FIG. 1 is a minimum stimulation intensity at which a training effect can be obtained. A training effect cannot be obtained at a stimulation intensity that is lower than the stimulation intensity S1.

It is assumed that electrical muscular stimulation is applied at a stimulation intensity that is equal to or lower than the sensitivity threshold in order to realize unconscious training in a waking state. Since the sensitivity threshold in a waking state is low, the electrical muscular stimulation need be suppressed low. However, a training effect cannot be obtained by electrical muscular stimulation of a low intensity.

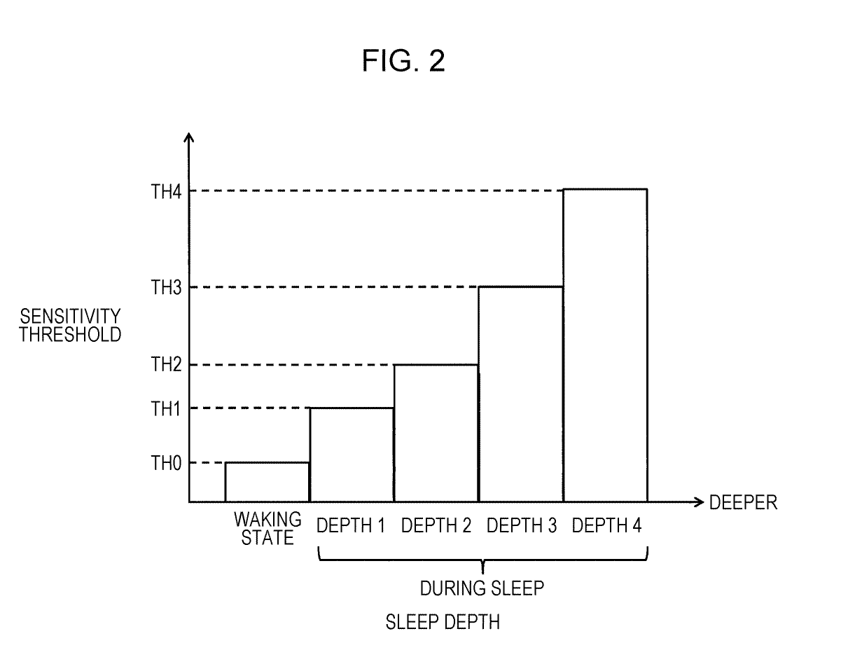

As disclosed in Japanese Unexamined Patent Application Publication No. 2010-200956, the sensitivity threshold changes in accordance with the degree of waking and is higher during sleep than during a waking state. Furthermore, as the sleep depth (also called a sleep stage) becomes deeper, the sensitivity threshold increases. FIG. 2 schematically illustrates a relationship between the sleep depth and the sensitivity threshold. FIG. 2 is a diagram obtained by modifying FIG. 2 of Japanese Unexamined Patent Application Publication No. 2010-200956 by the inventors of the present invention for convenience of understanding. In FIG. 2, the horizontal axis represents the sleep depth, and the vertical axis represents the sensitivity threshold. As is clear from FIG. 2, for example, the sensitivity threshold at the sleep depth 4 is much higher than that in a waking state.

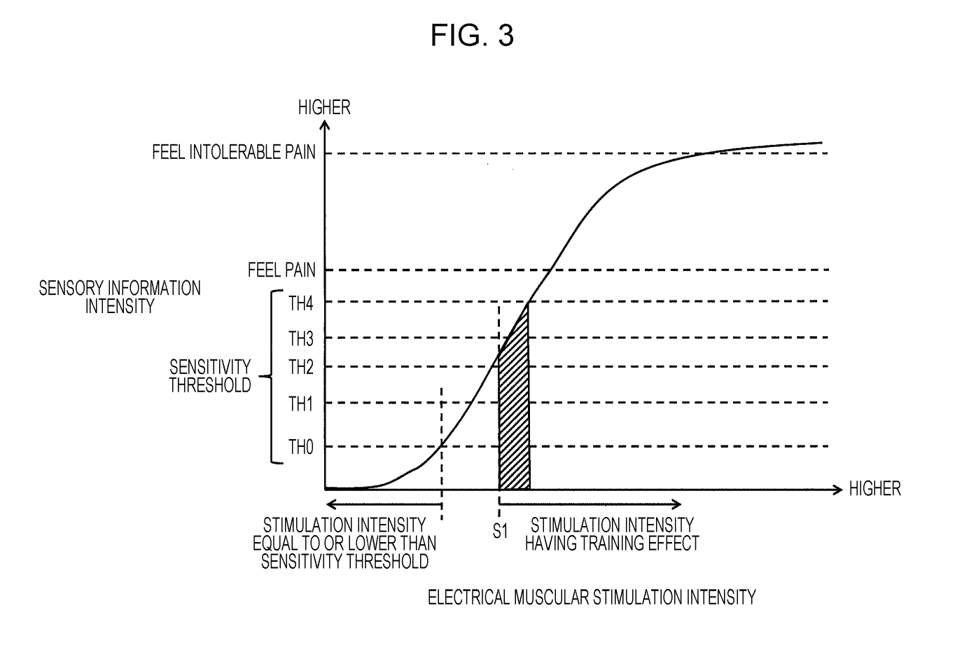

FIG. 3 virtually illustrates a relationship between the electrical muscular stimulation intensity and the sensitivity threshold at each sleep depth. Note that FIG. 3 is a combination of FIGS. 1 and 2. The sensitivity threshold and the degree of increase of the sensitivity threshold vary from one user to another. As illustrated in FIG. 3, it can be assumed that there are users who do not exceed the sensitivity threshold even upon application of electrical muscular stimulation of an intensity at which a training effect can be obtained during sleep at a deep sleep depth (e.g., the sleep depth 3 and the sleep depth 4 in FIG. 2).

However, this does not mean that electrical muscular stimulation of the intensity (S1) at which a training effect can be obtained can be always applied during sleep. In the case of a user illustrated in FIG. 3, if electrical muscular stimulation of S1 is applied at a sleep depth 1 and a sleep depth 2, the intensity of electrical muscular stimulation exceeds the sensitivity threshold, and user's sleep is hindered. Meanwhile, at the sleep depth 4, the sensitivity threshold is higher than S1, and therefore application of electrical muscular stimulation that is slightly higher than S1 is permitted. Specifically, electrical muscular stimulation of an intensity included in the region with diagonal lines in FIG. 3 may be applied. This makes it possible to improve the efficiency of training.

In view of this, the inventors of the present invention arrived at a technique of estimating a sleep depth of a user and adjusting an electrical muscular stimulation intensity in accordance with the estimated sleep depth.

One aspect of the present disclosure is an electrical stimulation apparatus including: a sleep depth determiner that determines a first sleep depth by using first biological information of a user; an electrical muscular stimulation intensity determiner that determines a first electrical muscular stimulation intensity on basis of the first sleep depth; and an output that outputs first electrical muscular stimulation by using electrodes arranged on a skin of the user at the first electrical muscular stimulation intensity.

The electrical stimulation apparatus may be arranged such that the sleep depth determiner that determines a second sleep depth by using second biological information of the user, the first sleep depth is a sleep depth before the first electrical muscular stimulation is given to the user and the second sleep depth is a sleep depth after the first electrical muscular stimulation is given to the user, and the electrical muscular stimulation intensity determiner changes the first electrical muscular stimulation intensity by comparing the first sleep depth and the second sleep depth.

The electrical stimulation apparatus may be arranged such that the electrical muscular stimulation intensity determiner determines to increase the first electrical muscular stimulation intensity in a case where the second sleep depth is equal to or deeper than the first sleep depth.

The electrical stimulation apparatus may be arranged to further include: a biological information measurer that measures biological information of the user including the first biological information and the second biological information, wherein the first biological information is measured before the first electrical muscular stimulation is given to the user and the second biological information is measured after the first electrical muscular stimulation is given to the user, and the first sleep depth being based on the first biological information and the second sleep depth being based on the second biological information.

The electrical stimulation apparatus may be arranged such that the second biological information is measured by the biological information measurer after elapse of a first period from output of the first electrical muscular stimulation.

The electrical stimulation apparatus may be arranged such that the first period is not less than 300 milliseconds and not more than 500 milliseconds.

The electrical stimulation apparatus may be arranged such that the biological information is related to a total number of body movements during a predetermined period.

The electrical stimulation apparatus may be arranged such that the electrical muscular stimulation intensity determiner determines to lower the first electrical muscular stimulation intensity in a case where the biological information of the user changes at second time intervals or shorter predetermined times or more after elapse of the first period from output of the first electrical muscular stimulation.

The electrical stimulation apparatus may be arranged such that the biological information is related to a brain wave.

In the embodiments below, a method for estimating a sleep depth and a method for adjusting an electrical muscular stimulation intensity are described in detail.

### Embodiment 1

A sleep-time electrical muscular stimulation apparatus (hereinafter referred to as an “electrical stimulation apparatus”) according to the present embodiment is described below. Note that each of the embodiments is mainly described by using functional blocks of the electrical stimulation apparatus. A hardware configuration for realizing each embodiment is described at the end of the embodiment. Note, however, that hardware may be referred to as needed for convenience of understanding.

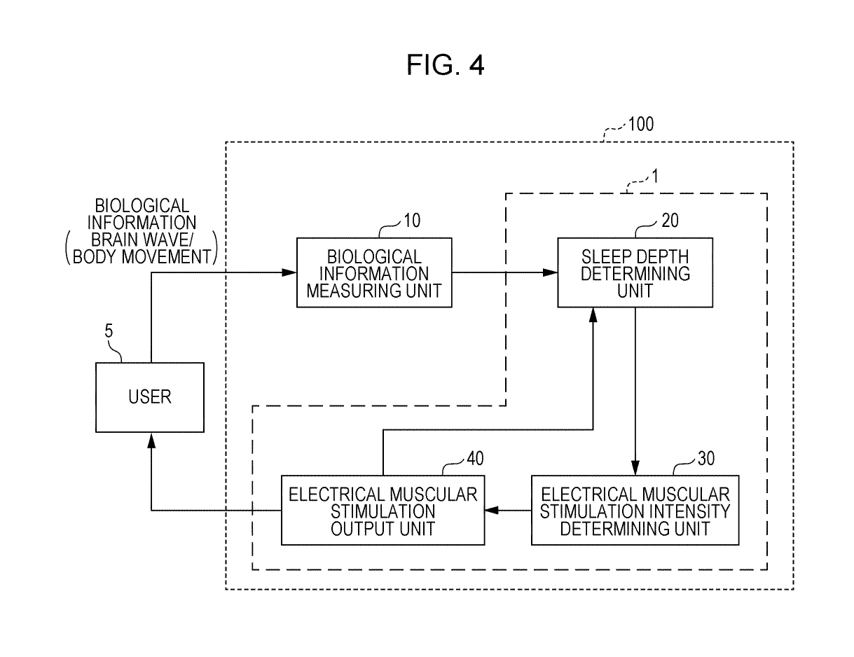

FIG. 4 illustrates a configuration of functional blocks of an electrical stimulation apparatus 100 according to the present embodiment. The electrical stimulation apparatus 100 includes a biological information measuring unit 10 and an electrical muscular stimulation device 1. The electrical muscular stimulation device 1 includes a sleep depth determining unit 20, an electrical muscular stimulation intensity determining unit 30, and an electrical muscular stimulation output unit 40. The electrical muscular stimulation device 1 and the biological information measuring unit 10 are connected to each other by a wire or by radio.

### Use Environment

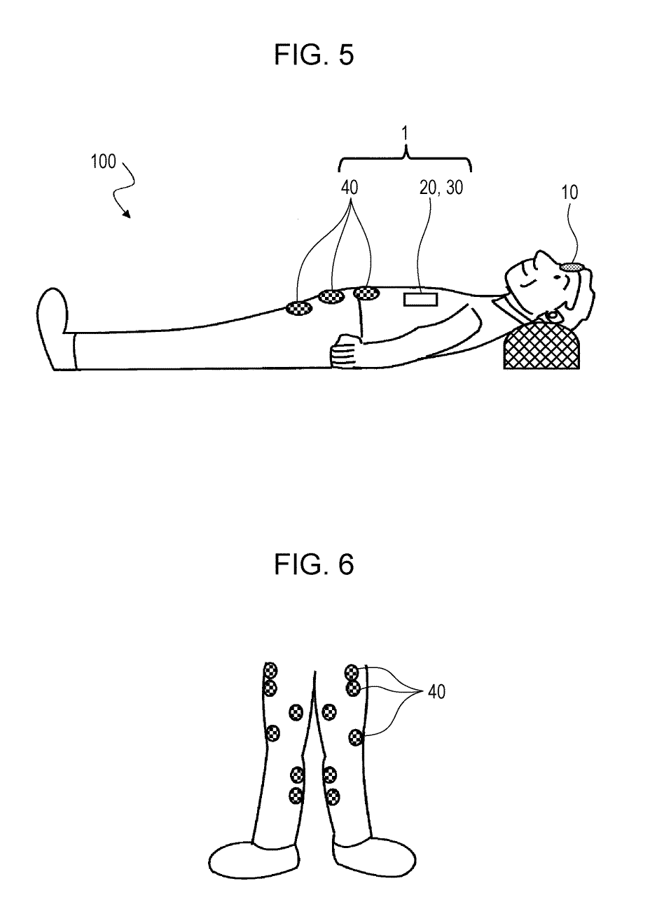

FIG. 5 illustrates an example of a configuration and a use environment of the electrical stimulation apparatus 100 according to the present embodiment. FIG. 5 is an apparatus suitably used for abdominal training. FIG. 6 is a simplified diagram of an apparatus suitably used for leg training. For convenience of description, only the electrical muscular stimulation output unit 40 is illustrated. Note that the electrical muscular stimulation output unit 40 is, for example, electrodes and a wire for feeding electric power to the electrodes. The electrical muscular stimulation output unit 40 may include a power source circuit (not illustrated). In either example, it is assumed that the electrodes are embedded in clothes.

The electrical stimulation apparatus 100 of FIG. 5 corresponds to the apparatus configuration of Embodiment 1 illustrated in FIG. 4. The biological information measuring unit 10 measures biological information of a user 5 during sleep and transmits measured data to the sleep depth determining unit 20. The biological information is, for example, a brain wave and/or body movement of the user 5. FIG. 5 illustrates an example in which the biological information measuring unit 10 measures a brain wave and transmits brain wave data to the sleep depth determining unit 20. In this example, the biological information measuring unit 10 can be an electroencephalograph.

The sleep depth determining unit 20 determines a sleep depth of the user 5 on the basis of biological information received from the biological information measuring unit 10 and transmits data indicative of a result of the determination to the electrical muscular stimulation intensity determining unit 30. The biological information measuring unit 10 and the sleep depth determining unit 20 are connected to each other by a wire or by radio. The electrical muscular stimulation intensity determining unit 30 and the electrical muscular stimulation output unit 40 are connected to each other by a wire. This allows electrical muscular stimulation of an intensity determined by the electrical muscular stimulation intensity determining unit 30 to be output from the electrical muscular stimulation output unit 40.

Details of each constituent element are described below.

### Biological Information Measuring Unit 10

The biological information measuring unit 10 is a circuit for measuring biological information of the user 5 by using a plurality of electrodes disposed on the user 5. The biological information measuring unit 10 can include the plurality of electrodes and an amplifier.

For example, in the following description, it is assumed that the biological information measuring unit 10 is a measuring instrument for measuring a brain wave of the user 5. In this case, an electroencephalograph is provided as the biological information measuring unit 10. The biological information measuring unit 10 has a plurality of electrodes. The plurality of electrodes include a measuring electrode and a reference electrode. The measuring electrode may be disposed at any of 19 portions defined in the ten-twenty electrode system. Alternatively, a single measuring electrode may be disposed on a frontal region of head including a forehead of the user 5. The reference electrode (reference) may be disposed on a left mastoid or a right mastoid.

The biological information measuring unit 10 measures a difference in electric potential between the plurality of electrodes as a brain wave of the user 5. The plurality of electrodes can include a ground. The ground may be disposed on the left mastoid or the right mastoid. The amplifier may amplify the measured difference in electric potential. The biological information measuring unit 10 transmits the brain wave of the user 5 amplified by the amplifier to the sleep depth determining unit 20.

The biological information measuring unit 10 may be a measuring instrument that measures body movement of the user 5. In this case, the biological information measuring unit 10 is a sensor, more specifically, a general motion sensor. It is unnecessary to perform detailed detection of user's movement, and it is only necessary to detect the presence of absence of body movement. For example, a pressure sensor may be used as the biological information measuring unit 10.

### Sleep Depth Determining Unit 20

The sleep depth determining unit 20 determines a sleep depth on the basis of biological information (e.g., brain wave) of the user 5 measured by the biological information measuring unit 10. Specifically, the sleep depth determining unit 20 determines a sleep stage corresponding to the biological information of the user 5 by referring to a first standard.

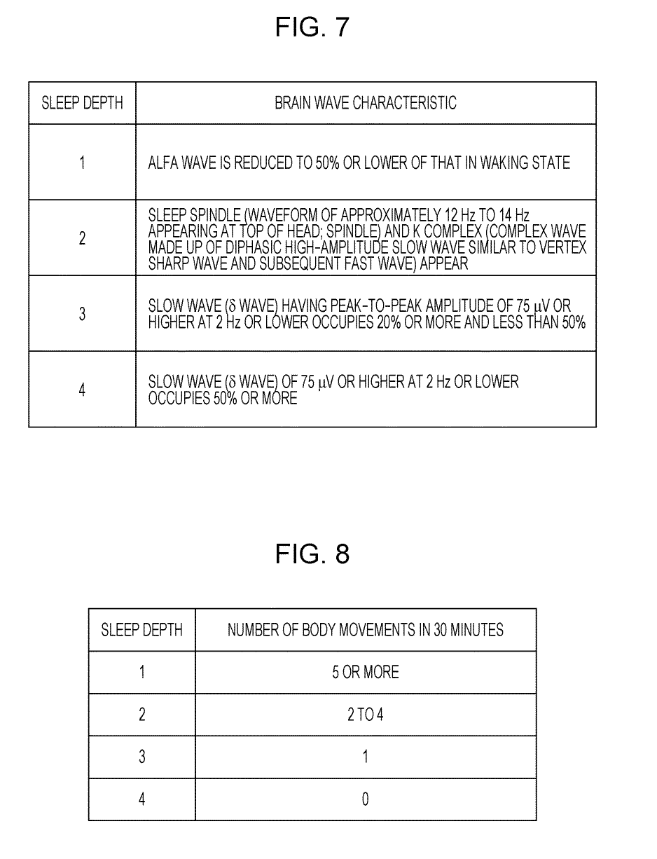

FIG. 7 illustrates an example of the first standard used in a case where the biological information is a brain wave. FIG. 7 illustrates a relationship between sleep depths and characteristics of a brain wave.

As is clear from description of a characteristic of a brain wave at the sleep depth 1, the amplitude of an alfa wave of the user 5 in a waking state need be found in advance. Furthermore, “75 μV” in the description of brain wave characteristics at the sleep depth 3 and the sleep depth 4 can vary depending on a measuring instrument or a measurement condition and can be changed in accordance with a used type of the biological information measuring unit 10 (electroencephalograph) and a measurement condition. Note that FIG. 7 merely illustrates an example.

In a case where the biological information is body movement, the sleep depth determining unit 20 may determine a sleep depth as follows. First, a sleep depth is estimated on the basis of the frequency of body movement of a predetermined level or higher. There is a correlation between a sleep depth and the frequency of occurrence of body movement, and the frequency of occurrence of body movement decreases as the sleep depth becomes deeper (KANBAYASHI, HAGIWARA, “An Approach on Estimation of Sleep Cycle Using Occurrence Rate of Body Movements”, 2011). The sleep depth can be determined, for example, on the basis of the number of occurrences of body movement in 30 minutes. Specifically, it is determined that the sleep depth is the sleep depth 4 in a case where the number of occurrences of body movement is 0, it is determined that the sleep depth is the sleep depth 3 in a case where the number of occurrences of body movement is 1, it is determined that the sleep depth is the sleep depth 2 in a case where the number of occurrences of body movement is 2 to 4, and it is determined that the sleep depth is the sleep depth 1 in a case where the number of occurrences of body movement is 5 or more. FIG. 8 illustrates a relationship between sleep depths and characteristics of body movement.

Furthermore, the sleep depth determining unit 20 receives information on a timing of output of electrical muscular stimulation from the electrical muscular stimulation output unit 40 and determines a sleep depth after the electrical muscular stimulation on the basis of a brain wave of the user 5 measured after output of the electrical muscular stimulation. In this case, the sleep depth determined before the electrical muscular stimulation is a sleep depth before the electrical muscular stimulation. The sleep depth determining unit 20 transmits information for specifying the determined sleep depth to the electrical muscular stimulation intensity determining unit 30.

### Electrical Muscular Stimulation Intensity Determining Unit 30

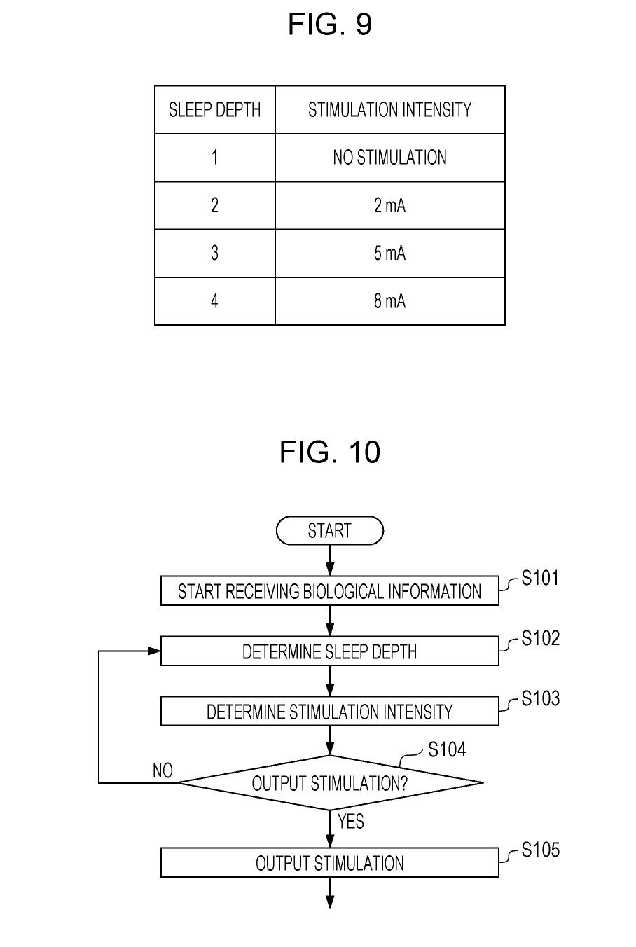

The electrical muscular stimulation intensity determining unit 30 determines an intensity of electrical muscular stimulation to be applied to the user 5 on the basis of the sleep depth of the user 5 determined by the sleep depth determining unit 20. Specifically, the electrical muscular stimulation intensity determining unit 30 determines the intensity of electrical muscular stimulation to be applied to the user 5 by referring to a second standard. The electrical muscular stimulation intensity is an electric current value in a case where a source of electrical muscular stimulation is an electric current source and is a voltage value in a case where a source of electrical muscular stimulation is a voltage source. FIG. 9 illustrates an example of the second standard. As illustrated in FIG. 9, in a case where the sleep depth is shallow, i.e., in a case where the sleep depth is 1, it is possible that no electrical muscular stimulation be output.

As described above, a sleep depth and a sensitivity threshold vary from one individual to another. It is therefore possible that a stimulation intensity that does not exceed a sensitivity threshold at each sleep depth be found in advance for each individual and the second standard be created in advance.

After electrical muscular stimulation is output at the determined electrical muscular stimulation intensity, the electrical muscular stimulation intensity determining unit 30 may change the electrical muscular stimulation intensity by comparing a sleep depth before the electrical muscular stimulation and a sleep depth after the electrical muscular stimulation. Specifically, in a case where the sleep depth after the electrical muscular stimulation is shallower than that before the electrical muscular stimulation, the electrical muscular stimulation intensity determining unit 30 changes the electrical muscular stimulation to one having a lower intensity. Meanwhile, in a case where the sleep depth after the electrical muscular stimulation is deeper than that before the electrical muscular stimulation (including a case where the sleep depth after the electrical muscular stimulation is the same as that before the electrical muscular stimulation), the electrical muscular stimulation intensity determining unit 30 changes the electrical muscular stimulation to one having a higher intensity.

Alternatively, the electrical muscular stimulation intensity determining unit 30 may determine to apply electrical muscular stimulation of a predetermined intensity or lower (e.g., 1 mA) that is a sufficiently low intensity and change the electrical muscular stimulation intensity by comparing a sleep depth before the electrical muscular stimulation and a sleep depth after the electrical muscular stimulation. Specifically, in a case where the sleep depth after the electrical muscular stimulation is shallower than that before the electrical muscular stimulation, the electrical muscular stimulation intensity determining unit 30 changes the electrical muscular stimulation to one having a lower intensity. Meanwhile, in a case where the sleep depth after the electrical muscular stimulation is deeper than that before the electrical muscular stimulation (including a case where the sleep depth after the electrical muscular stimulation is the same as that before the electrical muscular stimulation), the electrical muscular stimulation intensity determining unit 30 changes the electrical muscular stimulation to one having a higher intensity.

Note that in a case where the electrical muscular stimulation is changed by using the sleep depth before the electrical muscular stimulation and the sleep depth after the electrical muscular stimulation, the electrical muscular stimulation is changed by a value within a third predetermined range (e.g., 1 mA or more and 3 mA or less).

### Electrical Muscular Stimulation Output Unit 40

The electrical muscular stimulation output unit 40 applies electrical muscular stimulation at an intensity determined by the electrical muscular stimulation intensity determining unit 30 by using the electrodes for electrical muscular stimulation disposed on the user 5.

At least two electrodes, i.e., a positive electrode and a negative electrode are needed as the electrodes. The frequency of electrical muscular stimulation may be selected from a range of 4 Hz to 20 Hz. Alternatively, it is also possible to use a method of applying electrical muscular stimulation at a plurality of frequencies that interfere with each other in a living body. Then, timings of the start and end of the electrical muscular stimulation are transmitted to the sleep depth determining unit 20.

Next, a procedure of processes performed by the electrical muscular stimulation device 1 of the electrical stimulation apparatus 100 of FIG. 4 is described with reference to FIG. 10.

In Step S101, the biological information measuring unit 10 starts measurement of biological information of the user 5 and transmits the measured data of biological information to the sleep depth determining unit 20. The sleep depth determining unit 20 sequentially receives biological information transmitted from the biological information measuring unit 10 and records the biological information on a storage device (not illustrated).

In Step S102, the sleep depth determining unit 20 determines a sleep depth before application of electrical muscular stimulation on the basis of the biological information of the user 5 received from the biological information measuring unit 10. For example, the first standard that is referred to when a sleep stage is determined is one illustrated in FIG. 7 in a case where a brain wave is measured as a biological signal and is one illustrated in FIG. 8 in a case where body movement is measured as a biological signal. The sleep depth determining unit 20 transmits the determined sleep depth to the electrical muscular stimulation intensity determining unit 30.

In Step S103, the electrical muscular stimulation intensity determining unit 30 determines an electrical muscular stimulation intensity on the basis of the sleep depth received from the sleep depth determining unit 20. Note that since a sleep depth and a sensitivity threshold vary from one individual to another as described above, it is also possible that a stimulation intensity that does not exceed a sensitivity threshold at each sleep depth be found in advance for each individual and a value thus found be used.

In Step S104, the electrical muscular stimulation intensity determining unit 30 determines whether or not to output stimulation. This is because no stimulation is applied in a case where the sleep depth is 1 as illustrated in FIG. 9. In a case where stimulation is output, the process proceeds to Step S105, whereas in a case where no stimulation is output, the process returns to Step S102.

In Step S105, the electrical muscular stimulation output unit 40 applies electrical muscular stimulation to the user 5 at the intensity received from the electrical muscular stimulation intensity determining unit 30. The frequency of the electrical muscular stimulation may be selected from a range of 4 Hz to 20 Hz. Alternatively, it is also possible to use a method of applying electrical muscular stimulation at a plurality of frequencies that interfere with each other in a living body.

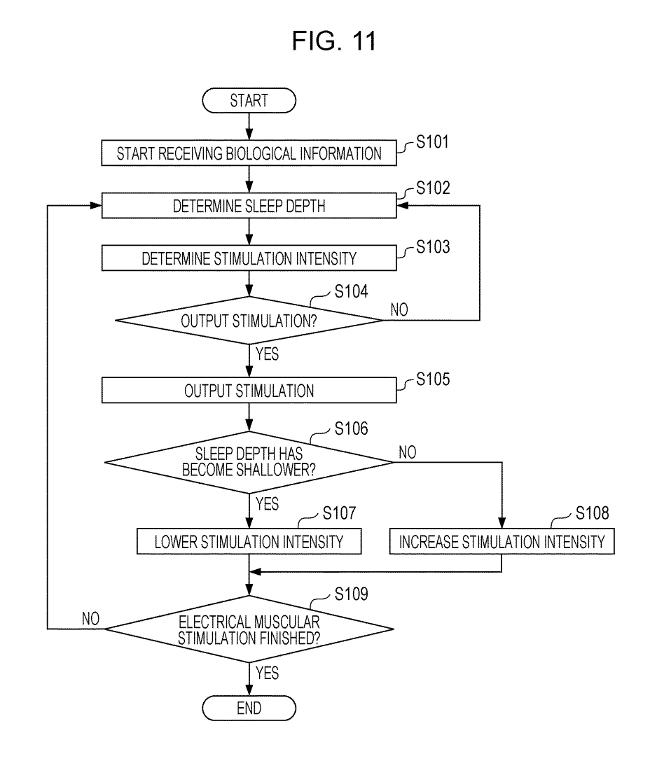

FIG. 10 is a procedure of basis processes of the electrical stimulation apparatus 100. An example of application of these processes is described below with reference to FIG. 11.

FIG. 11 illustrates a procedure of the example of application of the processes of the electrical muscular stimulation device 1 according to the present embodiment. Steps S101 to S105 are identical to those in FIG. 10, and therefore description thereof is omitted. Note that in the following description, it is assumed that, until the process in Step S105, the sleep depth determining unit 20 continues to store biological information received from the biological information measuring unit 10 and the sleep depth determining unit 20 sequentially determines a sleep depth.

In Step S106, the sleep depth determining unit 20 determines a sleep depth after stimulation output and determines whether or not the sleep depth has become shallower. In a case where the sleep depth has become shallower, the process proceeds to Step S107. Meanwhile, in a case where the sleep depth has not become shallower, i.e., in a case where the sleep depth remains the same or has become deeper, the process proceeds to Step S108. In order to determine a change in sleep depth, the sleep depth determining unit 20 stores at least information on the last sleep depth in a storage device (e.g., a buffer or a memory) (not illustrated). The sleep depth determining unit 20 transmits information on a newly-determined sleep depth to the electrical muscular stimulation intensity determining unit 30.

In Step S107, upon receipt of a result of determination indicating that the sleep depth has become shallower from the sleep depth determining unit 20, the electrical muscular stimulation intensity determining unit 30 lowers the stimulation intensity. Specifically, the electrical muscular stimulation intensity determining unit 30 lowers the stimulation intensity so that the stimulation intensity becomes lower than a sensitivity threshold corresponding to the newly-determined sleep depth. The process proceeds to Step S109.

In Step S108, the electrical muscular stimulation intensity determining unit 30 increases the stimulation intensity. Specifically, the electrical muscular stimulation intensity determining unit 30 increases the stimulation intensity so that the stimulation intensity becomes lower than a sensitivity threshold corresponding to the newly-determined sleep depth. The process proceeds to Step S109.

In Step S109, the electrical muscular stimulation intensity determining unit 30 determines whether or not to finish application of the electrical muscular stimulation. For example, a standard on which it is determined whether or not to finish application of the electrical muscular stimulation may be a case (FIG. 9) where the sleep depth has become 1 or may be a case where a period elapsed after the start of application of the electrical muscular stimulation has exceeded a predetermined value.

According to the electrical stimulation apparatus 100, a sleep depth of a user is determined on the basis of biological information (a brain wave or body movement), and electrical muscular stimulation is output at an intensity corresponding to the sleep depth. This makes it possible to apply electrical muscular stimulation at an intensity equal to or lower than a sensitivity threshold. It is therefore possible to realize efficient muscle training without hindering sleep of the user.

Furthermore, a sleep depth before electrical muscular stimulation and a sleep depth after the electrical muscular stimulation are compared with each other, and in a case where the sleep depth has become shallower, the stimulation intensity is lowered, whereas the stimulation intensity is increased in other cases. This makes it possible to continuously apply electrical muscular stimulation without user's consciousness without hindering user's sleep. It is therefore possible to further increase an effect of muscle training.

The inventors of the present invention think that measurement is easier in the example in which a sleep depth is determined by using body movement. This is because in the case of measurement of a brain wave, it is necessary to perform a troublesome operation of attaching electrodes on the head of a user. This does not deny use of a brain wave. It is technically possible to determine a sleep depth by using a brain wave.

The electrical stimulation apparatus 100 described above determines a sleep depth by using a brain wave or body movement as biological information. It is unnecessary to choose between a brain wave and body movement, and both of a brain wave and body movement can be used.

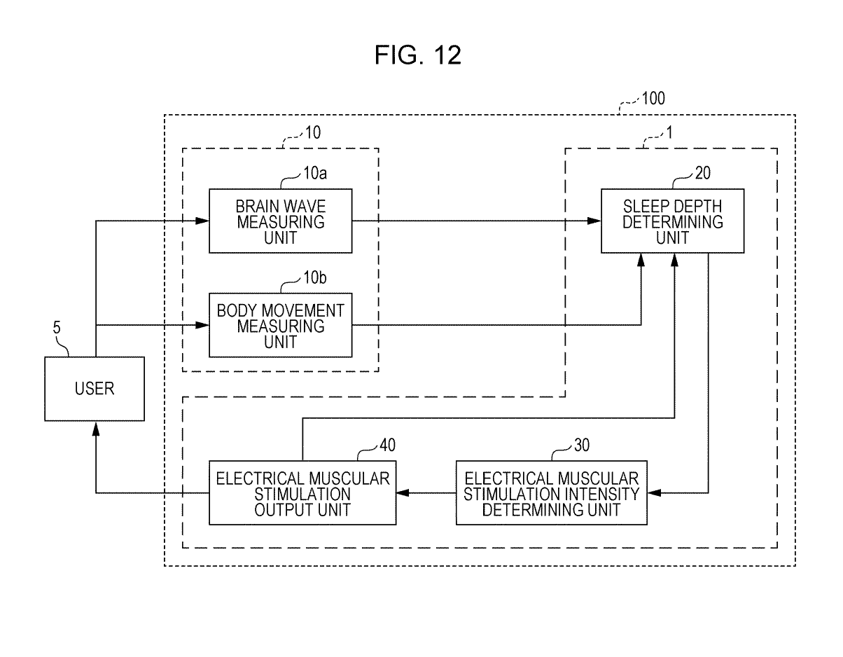

For example, FIG. 12 illustrates an electrical stimulation apparatus 100 including a biological information measuring unit 10 that is capable of measuring both of a brain wave and body movement. Constituent elements that have functions identical to those described above are given identical reference signs, and description thereof is omitted.

This biological information measuring unit 10 includes a brain wave measuring unit 10a that measures a brain wave and a body movement measuring unit 10b that measures body movement. The brain wave measuring unit 10a is a so-called electroencephalograph, and the body movement measuring unit 10b is a motion sensor. Results of measurement of the brain wave measuring unit 10a and the body movement measuring unit 10b are transmitted to the sleep depth determining unit 20.

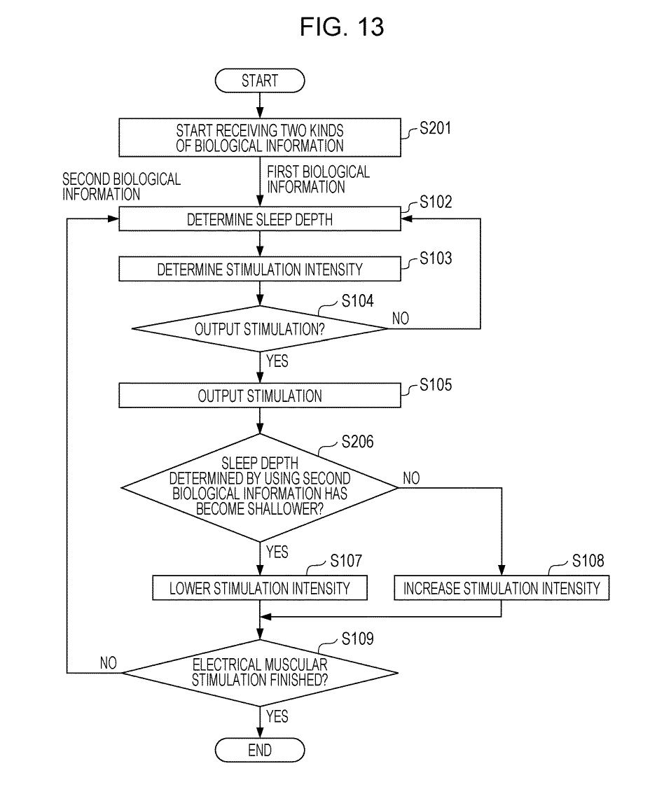

FIG. 13 illustrates a procedure of processes performed by the electrical muscular stimulation device 1 by using the two kinds of biological information. FIG. 13 is mainly different from FIG. 11 in that Steps S201 and S206 are provided instead of Steps S101 and S106 of FIG. 11. These differences and related changes are described below.

In Step S201, the sleep depth determining unit 20 starts receiving the two kinds of biological information of the user 5 measured by the biological information measuring unit 10, i.e., biological signals concerning both of a brain wave and body movement. In next Step S102, the sleep depth determining unit 20 determines a sleep depth by using one (e.g., a brain wave) of the two kinds of biological information.

Meanwhile, in later Step S206, the sleep depth determining unit 20 determines a sleep depth after stimulation output by using the other one (e.g., body movement) of the two kinds of biological information and then determines whether or not the sleep depth has become shallower than before.

Then, in processes in Step S102 and subsequent steps that are performed in a case where application of electrical muscular stimulation is not finished (in a case where the result in Step S109 is “NO”), the sleep depth determining unit 20 need just determine a sleep depth by using the other one (e.g., body movement) of the two kinds of biological information.

According to the aforementioned procedure, biological information used when stimulation is given for the first time and biological information used when stimulation is given for the second or subsequent time are different. Specifically, stimulation is given for the first time by using a brain wave, and stimulation is given for the second or subsequent time by using body movement. Alternatively, stimulation is given for the first time by using body movement, and stimulation is given for the second or subsequent time by using a brain wave. Since a plurality of kinds of biological information are used, even a lopsided result of one biological information can be compensated by the other biological information.

### Embodiment 2

In Embodiment 1, the electrical stimulation apparatus 100 (FIGS. 4 and 12) that determines a sleep depth by using a brain wave and/or body movement as biological information has been described. In the flow charts of FIGS. 11 and 13, it is determined whether or not a sleep depth has become shallower after stimulation output by using the biological information.

The inventors of the present invention conducted studies to more accurately determine a sleep depth after stimulation output and made a new finding.

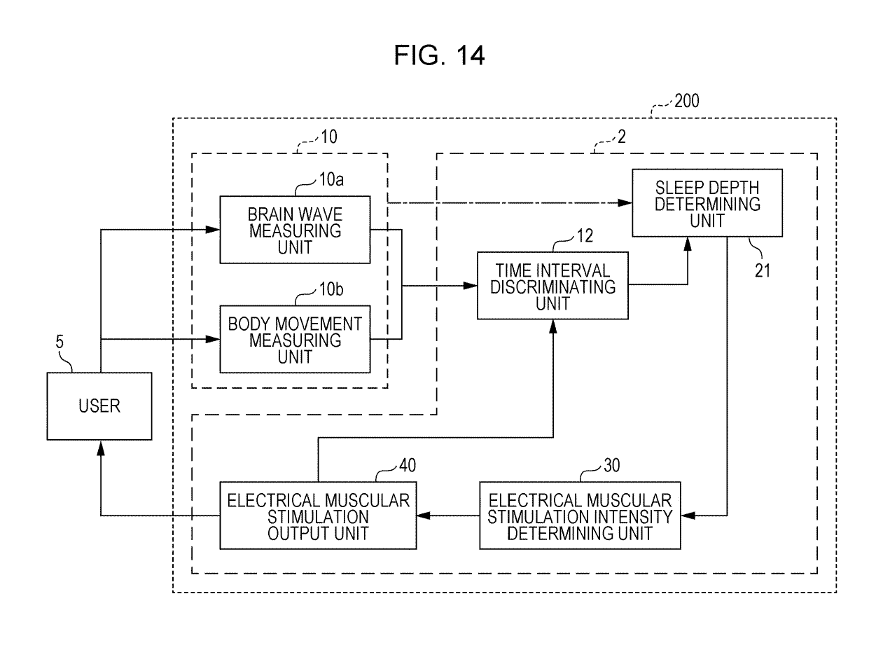

FIG. 14 illustrates a configuration of functional blocks of an electrical stimulation apparatus 200 according to the present embodiment. The electrical stimulation apparatus 200 is obtained by adding a time interval discriminating unit 12 to the configuration of FIG. 12. In the present embodiment, a sleep depth determining unit 21, an electrical muscular stimulation intensity determining unit 30, an electrical muscular stimulation output unit 40, and the time interval discriminating unit 12 constitute an electrical muscular stimulation device 2.

The time interval discriminating unit 12 receives biological information from a brain wave measuring unit 10a or a body movement measuring unit 10b of the biological information measuring unit 10. For example, the time interval discriminating unit 12 receives information on a brain wave of a user 5 from the brain wave measuring unit 10a. Alternatively, the time interval discriminating unit 12 receives information on the presence or absence of body movement of the user 5 from the body movement measuring unit 10b. Furthermore, the time interval discriminating unit 12 receives information on timings of the start and end of application (stimulation application period) of electrical muscular stimulation from the electrical muscular stimulation output unit 40.

The time interval discriminating unit 12 discriminates whether or not a time interval in which a brain wave or body movement changes is affected by the electrical muscular stimulation by using the received information. For example, in a time interval in which body movement changes, the time interval discriminating unit 12 discriminates whether or not the change of body movement is a voluntary action of the user 5 or an action induced by the electrical muscular stimulation. This process is sometimes referred to as a “time interval discriminating process”.

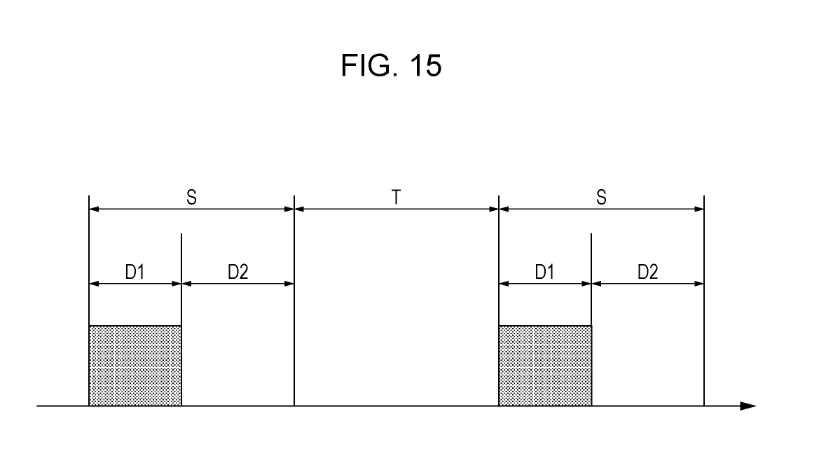

FIG. 15 schematically illustrates a discriminating method. In the present embodiment, a time interval D1 during which electrical muscular stimulation is being output and a subsequent predetermined period D2 are regarded as a time interval S that is being affected by the electrical muscular stimulation. A time interval between two adjacent time intervals S affected by electrical muscular stimulation is regarded as a time interval T that is not affected by electrical muscular stimulation.

The predetermined period D2 may be, for example, 300 milliseconds or may be 500 milliseconds. Specifically, in a case where a biological signal of the user 5 is detected in the time interval S by the biological information measuring unit 10, it is determined that the biological signal is affected by electrical muscular stimulation. Meanwhile, in a case where a biological signal of the user 5 is detected in the time interval T by the biological information measuring unit 10, it is determined that the biological signal is not affected by electrical muscular stimulation. Then, the time interval discriminating unit 12 transmits a result of the determination to the sleep depth determining unit 21.

The sleep depth determining unit 21 receives information on the result of the determination from the time interval discriminating unit 12 and receives biological information from the biological information measuring unit 10. The sleep depth determining unit 21 detects a change in sleep state of the user 5 by using not the biological signal measured during the time interval S that is affected by electrical muscular stimulation, but the biological signal measured during the time interval T that is not affected by electrical muscular stimulation by referring to the result of the determination received from the time interval discriminating unit 12. This allows the sleep depth determining unit 21 to accurately determine a sleep depth. The sleep depth determining unit 21 may determine a sleep depth by using a brain wave or may determine a sleep depth by using body movement. A method for determining a sleep depth is the same as that described in Embodiment 1.

Note that in a case where stimulation is given for the first time on the basis of a biological signal of the user 5, it is unnecessary for the time interval discriminating unit 12 to perform the time interval discriminating process. This is because it is clear that the biological signal is not affected by electrical muscular stimulation. The line with alternate long and short dashes extending from the biological information measuring unit 10 to the sleep depth determining unit 21 in FIG. 14 also means a process of bypassing the time interval discriminating unit 12.

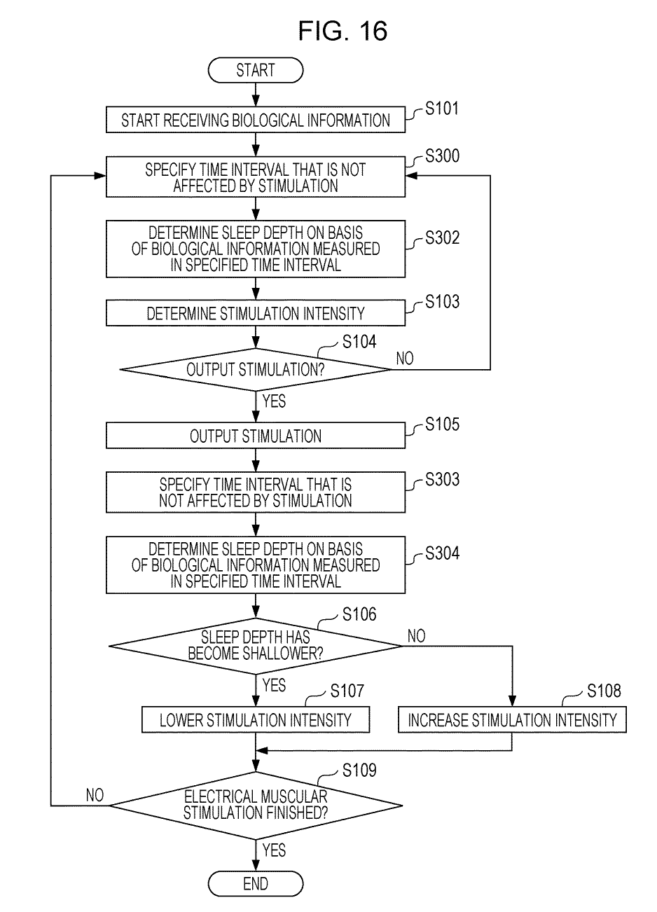

FIG. 16 illustrates a procedure of processes performed by the electrical muscular stimulation device 2 in consideration of a stimulation application period.

FIG. 16 is a flow chart obtained by replacing Step S102 in FIG. 11 with Steps S300 and S302 and adding Steps S303 and S304 to FIG. 11. These differences and related changes are described below.

First, in Step S101, biological information received by the sleep depth determining unit 21 may be a brain wave or may be body movement.

In Step S300, the time interval discriminating unit 12 specifies a time interval T that is not affected by stimulation by using information on a stimulation application period. Then, in Step S302, the sleep depth determining unit 21 determines a sleep depth by using biological information measured in the time interval T.

After stimulation output in Step S106, the time interval discriminating unit 12 specifies a time interval T in Step S303. Then, the sleep depth determining unit 21 determines a sleep depth by using biological information measured in the time interval T. The sleep depth determining unit 21 determines whether or not the sleep depth has become shallower in Step S106 by using a result of the determination.

According to the aforementioned processes, the sleep depth determining unit 21 detects a change in sleep state of the user 5 by using a biological signal measured in a time interval that is not affected by electrical muscular stimulation by using a result of a time interval discriminating process. This makes it possible to accurately determine a sleep depth of the user 5 even after stimulation output.

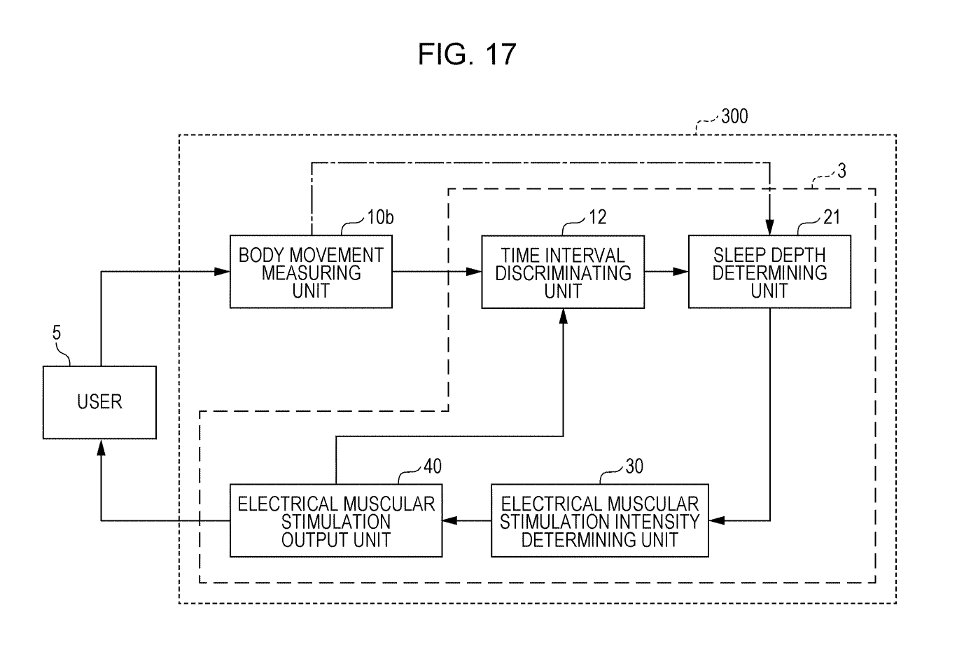

FIG. 17 illustrates a configuration of functional blocks of an electrical stimulation apparatus 300 according to a modification of the present embodiment. A difference of the electrical stimulation apparatus 300 from the electrical stimulation apparatus 200 illustrated in FIG. 14 is that body movement is obtained as a biological signal by a body movement measuring unit 10b. A reason why body movement is used as a biological signal is that the sensitivity threshold described above is mainly related to somatic sensation and therefore often appears as body movement. Accordingly, in a case where body movement is used as a biological signal, it is necessary to properly distinguish a time interval S that is affected by electrical muscular stimulation and a time interval T that is not affected by electrical muscular stimulation and to determine a sleep depth in the time interval T.

Note that body movement of a user can be largely classified into two kinds. One of the two kinds is an ordinary action during sleep such as roll-over. In this case, it can be said that a sleep state of the user has not changed. The other one of the two kinds is an action induced in response to electrical muscular stimulation. One example of such an action is waving aside an electrode part with a hand in an attempt of avoiding electrical muscular stimulation. In this case, it can be assumed that an electrical muscular stimulation intensity is equal to or higher than a waking threshold and the user is perceiving electrical muscular stimulation. This means that the electrical muscular stimulation can change a sleep state toward a shallower one. The former action occurs in a manner that is not in synchronization with electrical muscular stimulation, and therefore it is unlikely that body movement continues throughout the time intervals S and T. Meanwhile, the latter action, i.e., body movement induced in response to electrical muscular stimulation occurs in synchronization with electrical muscular stimulation. It may therefore be determined that a sleep state has changed in a case where body movement occurs in succession predetermined times or more every electrical muscular stimulation. The predetermined times may be two times or may be five times although the predetermined times vary depending on the frequency of electrical muscular stimulation.

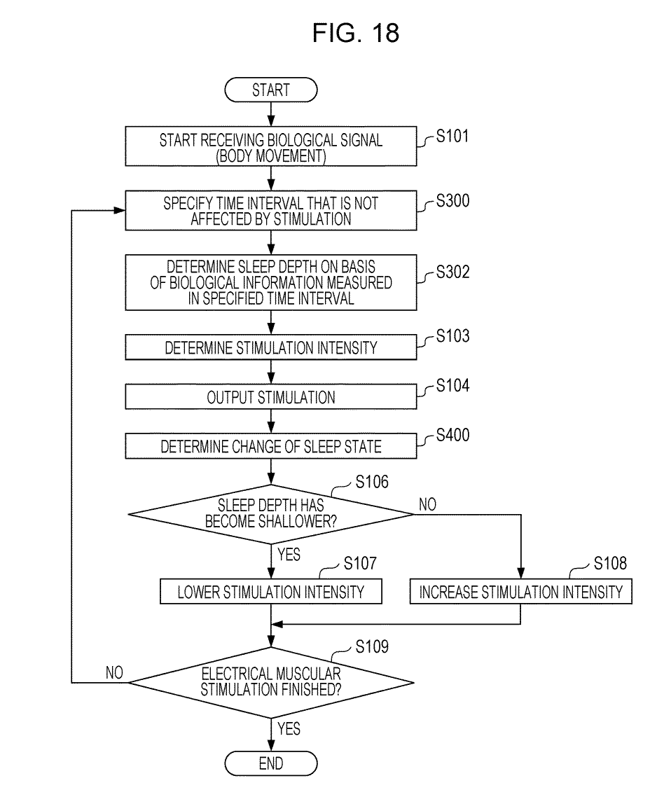

FIG. 18 illustrates a procedure of processes performed by an electrical muscular stimulation device 3 in consideration of a stimulation application period. Processes identical to those in FIG. 16 are given identical step numbers.

A difference of FIG. 18 from FIG. 16 is Step S400 of FIG. 18, which is actually a combination of Step S303 and S304 of FIG. 16.

That is, in Step S400, the time interval discriminating unit 12 discriminates between the time interval S that is affected by electrical muscular stimulation and the time interval T that is not affected by electrical muscular stimulation on the basis of body movement information of a user received from the body movement measuring unit 10b and information on a stimulation application period received from the electrical muscular stimulation output unit 40. A method of discrimination is the same as that described in association with FIG. 15. The sleep depth determining unit 21 determines a sleep depth by using biological information measured in the time interval T.

According to the electrical stimulation apparatus 300 of the present embodiment, it is possible to detect a sleep depth and a change thereof on the basis of a voluntary action of a user and thereby adjust an electrical muscular stimulation intensity. It is therefore possible to realize efficient muscle training without hindering sleep of the user.

Note that electrical muscular stimulation may be used as wake-up alarm. In this case, electrical muscular stimulation is output at an intensity higher than a sensitivity threshold when a preset time comes, and the electrical muscular stimulation intensity is increased in a case where a sleep depth does not change or becomes deeper.

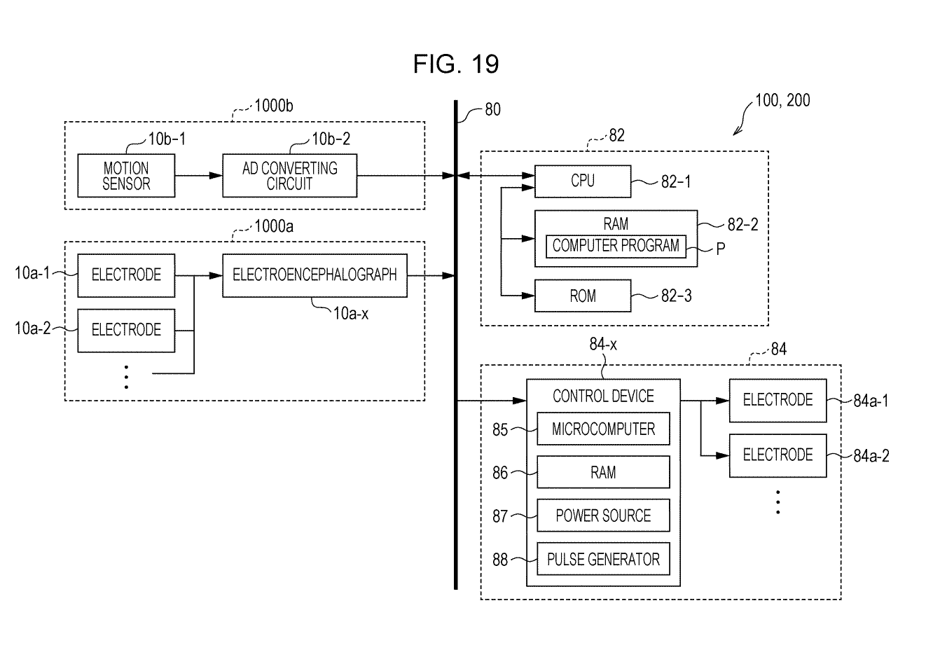

FIG. 19 illustrates a hardware configuration common to the electrical stimulation apparatuses 100 and 200 described above. Note that the configuration of the electrical stimulation apparatus 300 (FIG. 17) is obtained by removing elements concerning a brain wave from the electrical stimulation apparatus 200, and therefore description thereof is omitted.

Each of the electrical stimulation apparatuses 100 and 200 includes a brain wave measuring device 1000a, which is a brain wave measuring unit, a body movement measuring device 1000b, which is a body movement measuring unit, a bus 80, a computer system 82, and an electrical muscular stimulation device 84. The bus 80 allows the brain wave measuring device 1000a, the body movement measuring device 1000b, the computer system 82, and the electrical muscular stimulation device 84 to be communicably connected to one another.

The brain wave measuring device 1000a includes an electroencephalograph 10a-x and a plurality of electrodes (e.g., electrodes 10a-1 and 10a-2). The electroencephalograph 10a-x is not limited to a specific one, provided that the electroencephalograph 10a-x is a commonly-available one. Detailed description of the electroencephalograph 10a-x is omitted.

The body movement measuring device 1000b includes a motion sensor 10b-1 and an AD converting circuit 10b-2 that converts an analog signal output by the motion sensor 10b-1 into a digital signal. The motion sensor 10b-1 and the AD converting circuit 10b-2 can also be commonly-available ones. Detailed description of the motion sensor 10b-1 and the AD converting circuit 10b-2 is omitted.

The computer system 82 includes a central processing unit (CPU) 82-1, a random access memory (RAM) 82-2, and a read-only memory (ROM) 82-3. In the RAM 82-2, a computer program P loaded from the ROM 82-3 by the CPU 82-1 is stored. The computer system 82 can be realized as the electrical muscular stimulation devices 1 through 3 described above. The procedures of the processes of the electrical muscular stimulation devices 1 through 3 that have been described as flow charts can be realized by execution of the computer program P by the CPU 82-1.

The electrical muscular stimulation device 84 includes a control device 84-x and a plurality of electrodes (e.g., electrodes 84a-1 and 84a-2). The control device 84-x includes a microcomputer 85, a RAM 86, a power source 87, and a pulse generator 88. The control device 84-x is also commonly available, and therefore description thereof is omitted.

The following describes, as an example, how the processes in FIGS. 10 and 11 are performed.

The CPU 82-1 of the computer system 82 determines a sleep depth on the basis of data of a biological signal received from the brain wave measuring device 1000a and/or the body movement measuring device 1000b by referring to the first standard (FIG. 7 or 8) and determines a stimulation intensity to be applied to the user 5 on the basis of the sleep depth by referring to the second standard (FIG. 9). The CPU 82-1 of the computer system 82 transmits a command value to the microcomputer 85 of the electrical muscular stimulation device 84. The microcomputer 85 controls an electric current value to be fed to the electrodes in accordance with the command value.

In a case where it is determined that a sleep depth after stimulation output has become shallower, the CPU 82-1 transmits a command value indicative of a lowered stimulation intensity to the microcomputer 85 by referring to the second standard (FIG. 9). The microcomputer 85 applies stimulation to the user 5 by passing an electric current of a smaller electric current value in accordance with the command value. Meanwhile, in a case where it is determined that the sleep depth after stimulation output has not changed or has become deeper, the CPU 82-1 transmits a command value indicative of a higher stimulation intensity to the microcomputer 85 by referring to the second standard (FIG. 9). The microcomputer 85 applies stimulation to the user 5 by passing an electric current of a larger electric current value in accordance with the command value.

An electrical stimulation apparatus described in each of the above embodiments is realized by executing a computer program by using the aforementioned hardware.

An electrical stimulation apparatus according to the present disclosure estimates a sleep depth of a user and adjusts an electrical muscular stimulation intensity on the basis of the estimated sleep depth. It is therefore possible to realize efficient muscle training without hindering sleep of a user. For example, the electrical stimulation apparatus according to the present disclosure is applicable to muscle maintenance of elderly people, muscle enhancement of athletes, and the like.

What is claimed is:

1. An electrical stimulation apparatus, comprising:
a sleep depth determiner that determines a first sleep depth by using first biological information of a user;
an electrical muscular stimulation intensity determiner that determines a first electrical muscular stimulation intensity on basis of the first sleep depth; and
an output that outputs first electrical muscular stimulation by using electrodes arranged on a skin of the user at the first electrical muscular stimulation intensity.

2. The electrical stimulation apparatus according to claim 1, wherein
the sleep depth determiner that determines a second sleep depth by using second biological information of the user,
the first sleep depth is a sleep depth before the first electrical muscular stimulation is given to the user and the second sleep depth is a sleep depth after the first electrical muscular stimulation is given to the user, and
the electrical muscular stimulation intensity determiner changes the first electrical muscular stimulation intensity by comparing the first sleep depth and the second sleep depth.

3. The electrical stimulation apparatus according to claim 2, wherein
the electrical muscular stimulation intensity determiner determines to increase the first electrical muscular stimulation intensity in a case where the second sleep depth is equal to or deeper than the first sleep depth.

4. The electrical stimulation apparatus according to claim 2, further comprising:
a biological information measurer that measures biological information of the user including the first biological information and the second biological information,
wherein the first biological information is measured before the first electrical muscular stimulation is given to the user and the second biological information is measured after the first electrical muscular stimulation is given to the user, and
the first sleep depth being based on the first biological information and the second sleep depth being based on the second biological information.

5. The electrical stimulation apparatus according to claim 4, wherein
the second biological information is measured by the biological information measurer after elapse of a first period from output of the first electrical muscular stimulation.

6. The electrical stimulation apparatus according to claim 5, wherein
the first period is not less than 300 milliseconds and not more than 500 milliseconds.

7. The electrical stimulation apparatus according to claim 4, wherein
the biological information is related to a total number of body movements during a predetermined period.

8. The electrical stimulation apparatus according to claim 7, wherein
the electrical muscular stimulation intensity determiner determines to lower the first electrical muscular stimulation intensity in a case where the biological information of the user changes at second time intervals or shorter predetermined times or more after elapse of the first period from output of the first electrical muscular stimulation.

9. The electrical stimulation apparatus according to claim 4, wherein
the biological information is related to a brain wave.

10. An electrical stimulation method, comprising:
(a) determining a first sleep depth by using first biological information of a user;
(b) determining a first electrical muscular stimulation intensity on basis of the first sleep depth; and
(c) outputting first electrical muscular stimulation by using electrodes arranged on a skin of the user at the determined electrical muscular stimulation intensity.

11. The electrical stimulation method according to claim 10, further comprising:
(d) determining a second sleep depth by using second biological information of the user, the first sleep depth being a sleep depth before the first electrical muscular stimulation is given to the user, the second sleep depth being a sleep depth after the first electrical muscular stimulation is given to the user,
(e) changing the first electrical muscular stimulation intensity by comparing the first sleep depth and the second sleep depth.

12. The electrical stimulation method according to claim 10, further comprising:
(f) increasing the first electrical muscular stimulation intensity in a case where the second sleep depth is equal to or deeper than the first sleep depth.

13. The electrical stimulation method according to claim 11, further comprising:
(g) measuring biological information of the user including the first biological information and the second biological information,
wherein the first biological information is measured before the first electrical muscular stimulation is given to the user and the second biological information is measured after the first electrical muscular stimulation is given to the user,
the first sleep depth being based on the first biological information and the second sleep depth being based on the second biological information.

14. The electrical stimulation method according to claim 10, wherein
the second biological information is measured after elapse of a first period from output of the first electrical muscular stimulation.

15. The electrical stimulation apparatus according to claim 10, wherein
the first period is not less than 300 milliseconds and not more than 500 milliseconds.

16. The electrical stimulation method according to claim 13, wherein
the biological information is related to a total number of body movements during a predetermined period.

17. The electrical stimulation method according to claim 16, further comprising:
(h) determining to lower the first electrical muscular stimulation intensity in a case where the biological information of the user changes at second time intervals or shorter predetermined times or more after elapse of the first period from output of the first electrical muscular stimulation.

18. The electrical stimulation method according to claim 13, wherein
the biological information is related to a brain wave.

19. A non-transitory computer-readable recording medium storing a control program for causing an apparatus including a processor to execute processes including:
(a) determining a first sleep depth by using first biological information of a user;
(b) determining an first electrical muscular stimulation intensity on basis of the first sleep depth; and
(c) outputting first electrical muscular stimulation by using electrodes arranged on a skin of the user at the determined electrical muscular stimulation intensity.

20. The non-transitory computer-readable recording medium according to claim 19, wherein
the processes further include:
(d) determining a second sleep depth by using second biological information of the user, the first sleep depth being a sleep depth before the first electrical muscular stimulation is given to the user, the second sleep depth being a sleep depth after the first electrical muscular stimulation is given to the user,
(e) changing the first electrical muscular stimulation intensity by comparing the first sleep depth and the second sleep depth.

21. The non-transitory computer-readable recording medium according to claim 19, wherein
the processes further include:
(f) measuring biological information of the user including the first biological information and the second biological information,
wherein the first biological information is measured before the first electrical muscular stimulation is given to the user and the second biological information is measured after the first electrical muscular stimulation is given to the user,
the first sleep depth being based on the first biological information and the second sleep depth being based on the second biological information.

22. The non-transitory computer-readable recording medium according to claim 19, wherein
the second biological information is measured after elapse of a first period from output of the first electrical muscular stimulation.

23. The non-transitory computer-readable recording medium according to claim 21, wherein
the biological information is related to a total number of body movements during a predetermined period.

24. The non-transitory computer-readable recording medium according to claim 23, wherein
the processes further include:
(g) determining to lower the first electrical muscular stimulation intensity in a case where the biological information of the user changes at second time intervals or shorter predetermined times or more after elapse of the first period from output of the first electrical muscular stimulation.

25. The non-transitory computer-readable recording medium according to claim 21, wherein
the biological information is related to a brain wave.
